(19) United States
(12) Patent Application Publication
(10) Pub. No.: US 2018/0080928 A1
(43) Pub. Date: Mar. 22, 2018

(54) Method and System for Analyte Sensing

(71) Applicant: Assaf Cohen, Singapore (SG)

(72) Inventor: Assaf Cohen, Singapore (SG)

(21) Appl. No.: 15/348,029

(22) Filed: Nov. 10, 2016

Related U.S. Application Data
(63) Continuation of application No. PCT/IB2015/053482, filed on May 12, 2015.

(30) Foreign Application Priority Data
May 12, 2014 (SG) 10201402275U

(51) Int. Cl. G01N 33/543; C12Q 1/68; B01L 3/00

(52) U.S. Cl. CPC G01N 33/54326; G01N 33/54366; G01N 33/54353; C12Q 1/6834; C12Q 1/6825; B01L 3/502715; B01L 2400/043; B01L 3/50273; B01L 2300/0681; B01L 2300/0627; B01L 2200/10; B01L 2200/0668; B01L 3/502761

(57) ABSTRACT

The present application relates to a field of sensing and diagnostics, in general, and to a method for automatic on-site measurements of toxins, pathogens, heavy metals, explosives and any other analytes of interest.

### TECHNICAL FIELD

The present application relates to a field of sensing and diagnostics, in general, and to a method for automatic on-site measurements of toxins, pathogens, heavy metals, explosives and any other analytes of interest.

### BACKGROUND

Development of rapid, portable, sensitive, inexpensive and reusable molecular sensors has been of growing importance. Amongst the numerous sensors including, for example, optical, electrochemical and piezoelectric, magnetic-based sensors utilizing magnetic particles have gained popularity over recent years due to their unique properties allowing for efficient target capture and separation, relatively high sensitivity, and signal amplification.

The aforementioned magnetic particles have found wide usage in many sensing applications, such as in-vitro assays, cell sorting and target-specific MR contrast imaging. Easy surface functionalization of magnetic particles makes them attractive for use as labels and probes in biosensing applications. U.S. Pat. No. 4,452,773 relates to colloidal sized particles composed of magnetic iron oxide (Fe3O4) coated with a polysaccharide, preferably dextran, or a derivative thereof having pendant functional groups. The particles are used to label and separate cells, cellular membranes and other biological particles and molecules by means of a magnetic field.

U.S. Pat. No. 8,420,055 mentions amine functionalized magnetic nanoparticle compositions and processes for their preparation. These nanoparticles coated with various biomolecules are used in vitro assays, cell sorting applications and target specific MR contrast imaging.

Various mechanisms have been used to report the presence of magnetic particle-based labels and probes. Ferreira H A et al, “Biodetection using magnetically labelled molecules and arrays of spin valve sensors”, Journal of Applied Physics, Vol. 93, no. 10, 2004, pages 7281-7286, reports an on-chip spin-valve to detect surface binding of magnetic particle labels; WO 2005/010543 and WO 2005/010542 A2 mention the use of a Giant-Magneto-Resistance (GMR) sensor for detection of stray fields generated by magnetized particles. US 2008/0309329 A1 relates to a magneto-resistive sensor for detection of a magnetic field arising from the presence of magnetic particles. U.S. Pat 2010/0259254 A1 mentions a microfluidic device comprising a magnetic field generator and GMR for detection of stray fields generated by magnetized beads used as labels for biological molecules.

US 20080206104 A1 provides a method, a device and a system for determining the concentration of analytes in a fluid containing polarizable of polarized magnetic labels applied to biomolecular diagnostics.

US 20090170212 A1 relates to methods and (bio)sensor systems based on magnetic particles which are transported laterally under magnetic fields over a sensor surface with analyte specific probes. US 20110050215 A1 mentions a magnetic system for biosensors or a biosystem, wherein magnetic particles, which interact with molecules, are brought into a magnetic field, in order to be influenced via magnetic attraction or repulsion forces.

### SUMMARY

The present application describes the following embodiments:

### Embodiment 1

A method for sensing an analyte in a flow system using magnetic beads as sensing species, comprising the following steps:
a) pumping the flow containing said analyte into said flow system;
b) optionally filtering off magnetic impurities from the flow;
c) directing the flow into one or more reaction cartridges of said flow system for reacting the analyte with a binding entity attached to said magnetic beads inside the reaction cartridges, thereby releasing said magnetic beads from the reaction cartridges into the flow;
d) accumulating said flowing magnetic beads in a collecting area; and
e) recording a signal corresponding to the rate of the magnetic beads' release from said reaction cartridges (the amount of said accumulated magnetic beads per unit of time);
   wherein:
(i) said magnetic beads are initially attached to the surfaces of said reaction cartridges via a complex with said binding entity prior to reaction of said analyte with said binding entity;
(ii) said analyte is capable of reacting with said binding entity onto said magnetic beads, thereby replacing said magnetic beads from said complex and releasing them from said reaction cartridges into the flow,
(iii) said cartridges can operate in sequence or in parallel, and
(iv) said rate of the magnetic beads' release from said cartridges is proportional to the amount of said analyte bound in said reaction cartridge.

### Embodiment 2

The method according to embodiment 1, wherein steps a) to d) are continuously repeated for the predetermined amount of time, thereby amplifying the recorded signal.

### Embodiment 3

The method according to embodiment 1 further comprising one or more optional secondary amplification steps or magnetic cascade.

### Embodiment 4

The method according to embodiment 1, wherein said flowing magnetic beads are collected by utilizing magnetic field, physical barrier or chemical linkage.

### Embodiment 5

The method according to embodiment 1, wherein said signal is recorded with a magnetometer or with mass scales mounted onto said reaction cartridge or placed in a secondary location and measuring an applied force on a magnet attracting said magnetic beads.

### Embodiment 6

The method according to embodiment 1, wherein the amount of said accumulated magnetic beads is measured by using a magnetic properties sensor, said mass scales, or by applying a magnetic field to attract said magnetic beads and measure said force on a surface they apply for counting them.

### Embodiment 7

The method according to embodiment 1, wherein (1) the analyte is selected from toxins, viruses, pathogens, explosives or any other ecologically, agriculturally, forensically, toxically, therapeutically or pharmaceutically important molecules; and (2) the flow is any suitable liquid, gas or air.

### Embodiment 8

The method according to embodiment 7, where the liquid is water.

### Embodiment 9

The method according to embodiment 1, wherein the magnetic beads are paramagnetic beads, superparamagnetic beads, superferromagnetic beads, ferromagnetic beads or miniaturized magnets, all of which can be either non-magnetized or magnetized.

### Embodiment 10

The method according to embodiment 9, where said magnetic beads are ferromagnetic beads.

### Embodiment 11

The method according to embodiment 10, wherein said magnetic beads comprising a magnetic metal alloy core and a non-magnetic polymer shell, wherein said non-magnetic polymer shell is suitable (1) for adding surface functional groups to said magnetic beads for protecting said magnetic beads from an external media, and (2) for surface chemical attachment of the binding entity.

### Embodiment 12

The method according to embodiment 11, wherein the non-magnetic polymer shell is made of agarose, cellulose, porous glass or silica.

### Embodiment 13

The method according to embodiment 9, wherein said magnetic beads are non-magnetized beads, capable of being converted to permanent micro-magnets after being accumulated in the collecting area.

### Embodiment 14

The method according to claim 1, wherein said magnetic beads have a diameter in the range of 1 nm to 1 mm.

### Embodiment 15

The method according to claim 1, wherein (1) said magnetic beads are coated with said binding entity and attached to the surfaces of said reaction cartridges via affinity interactions with the immobilized analyte or analyte-analogue molecules, or (2) said magnetic beads are coated with the analyte or analyte-analogue molecules, and the walls of said reaction cartridge are coated with said binding entity.

### Embodiment 16

The method according to claim 1, wherein (1) the surfaces of said reaction cartridges are pre-activated with functional groups or functionalized with said analyte or analyte-analogue molecules, capable of reacting with said binding entity, using cross-linkers; or (2) the surfaces of said reaction cartridges are polymeric and pre-activated using carbene or nitrene chemistry.

### Embodiment 17

The method according to embodiment 16, wherein (1) said functional groups are selected from carboxyl, amine, N-hydroxysuccinimide (NHS), sulfhydryl, epoxide, hydroxyl and tosyl; (2) said functional groups are activated for coupling with the binding entity using the EDC-coupling chemistry for carboxylates, or glutaraldehyde for amines; or (3) said functional groups are activated for coupling with the binding entity using surface tosyl, cyanogen bromide, NHS or epoxide groups.

### Embodiment 18

The method according to embodiment 1, wherein said binding entity is a macromolecule, biomolecule or any other entity capable of specifically recognizing the analyte in the flow, selected from aptamers, nucleic acids, DNA, RNA, dsRNA cleavage system, oligonucleotides, polymers, imprinted polymers, antibodies, antibody fragments, antigens, enzymes, proteins or phage displays, or combination thereof.

### Embodiment 19

The flow system according to embodiment 1, whereas said magnetic beads or said analyte are (1) modified with one or more fluorescent probes for providing further information on said analyte; or (2) functionalized with an enzyme that can degrade a linker of secondary magnetic beads.

Various embodiments of the invention may allow various benefits, and may be used in conjunction with various applications. The details of one or more embodiments are set forth in the accompanying figure and the description below. Other features, objects and advantages of the described techniques will be apparent from the description and drawings and from the claims.

### BRIEF DESCRIPTION OF THE DRAWINGS

The present invention will be understood and appreciated more fully from the following detailed description taken in conjunction with the appended drawings.

FIG. 1 is a schematic diagram demonstrating the method according to one embodiment of the present invention.

FIG. 2 is a schematic diagram demonstrating the method according to another embodiment of the present invention, including the secondary amplification (or cascade).

FIG. 3 is the block diagram showing the electronics of the device.

FIG. 4A is a perspective view of the sensing device (Example 1) of the present invention.

FIG. 4B is a side view of the sensing device (Example 1) of the present invention.

FIG. 4C is a cross-sectional front view (A-A) of the sensing device (Example 1) of the present invention.

FIG. 5A is a perspective view of the sensing device (Example 2) of the present invention.

FIG. 5B is a side view of the sensing device (Example 2) of the present invention.

FIG. 5C is a cross-sectional front view (A-A) of the sensing device (Example 2) of the present invention.

FIG. 6A is a perspective view of the sensing device (Example 3) of the present invention.

FIG. 6B is a side view of the sensing device (Example 3) of the present invention.

FIG. 6C is a cross-sectional front view (A-A) of the sensing device (Example 3) of the present invention.

FIG. 7A is a perspective view of the sensing device (Example 4) of the present invention for simultaneous sensing of multiple analytes.

FIG. 7B is a front view of the sensing device (Example 4) of the present invention for simultaneous sensing of multiple analytes.

FIG. 8 is an illustration of the exemplary system of an embodiment.

FIG. 9 is a schematic diagram demonstrating the method according to one embodiment of the present invention.

FIG. 10 schematically shows another exemplary system of an embodiment.

FIG. 11 schematically shows another exemplary system of an embodiment.

### DETAILED DESCRIPTION

In the following description, various embodiments of the invention will be described. For purposes of illustration, specific configurations and details are set forth in order to provide a thorough understanding of the invention. However, it will be apparent to one skilled in the art that the invention is not limited to the specific details presented herein. Furthermore, well-known features may be omitted or simplified in order not to obscure the invention.

Amplification of the signal obtained from magnetic beads circulated in a flow system by their accumulation in a magnetic field is in the core of the present invention. Such accumulation can also be achieved through a magnetic attraction, chemical or physical reaction, enzymatic reaction physical separation of the beads and the like.

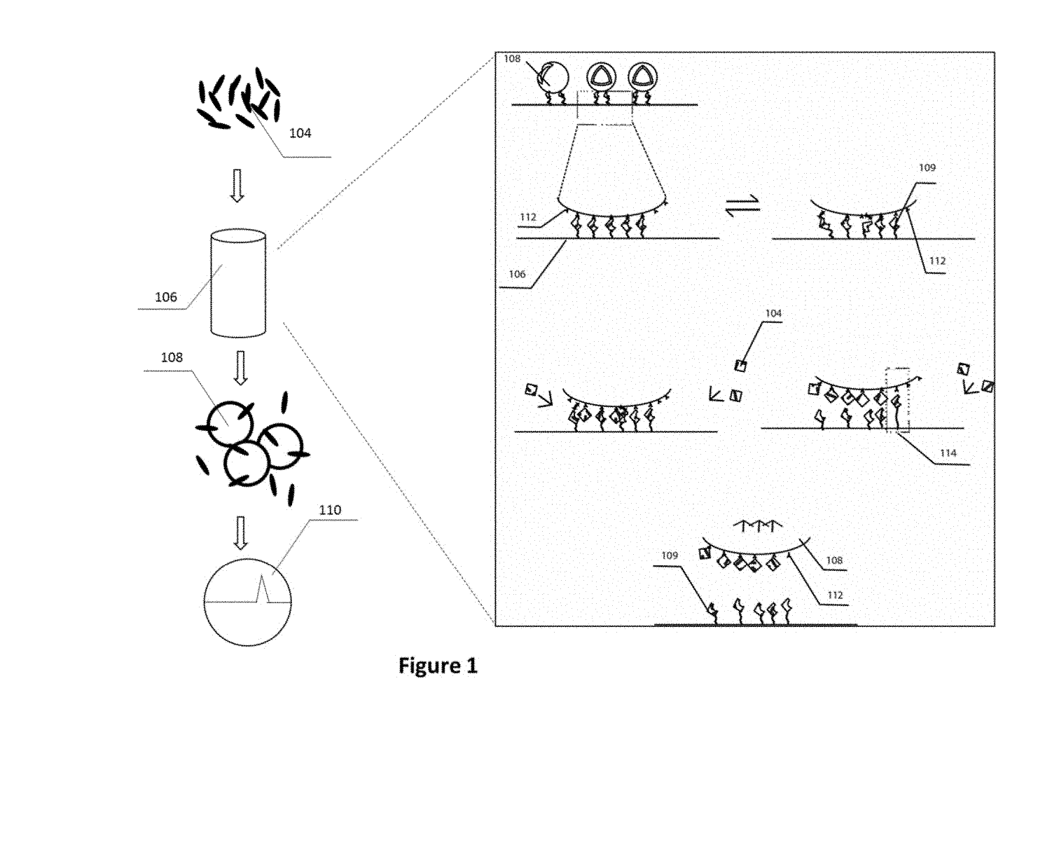

Reference is now made to FIG. 1 schematically showing the method according to one embodiment of the present invention. Analyte molecules 104 inside liquid flow 102 or any other carrying media, such as air or gas, passing cartridge 106 are subjected to the reaction with matching binding entity 112 inside the cartridge. Inner walls or surface of cartridge 106 from inside is functionalized with either native analyte 104 or analyte-analogue molecules 109, which are also capable of reacting with binding entity 112. Analyte-analogue molecules 109 normally have lower affinity to binding entity 112 than analyte 104. Different prior-art techniques may be used for surface functionalization of cartridge 106, followed by immobilization of molecules 104 or 109 using different cross-linkers. For example, surface functional groups may include carboxyl, amine, N-hydroxysuccinimide (NHS), sulfhydryl, epoxy, hydroxyl, and tosyl. Functional groups can be activated for coupling using, for example, the EDC-coupling chemistry for carboxylates, or glutaraldehyde for amines, in order to attach them to appropriate functional group of molecules 104 or 109. Alternatively, surface tosyl, cyanogen bromide, NHS-activated and epoxide groups may be used to attach the molecules directly without cross-linking agents.

In one embodiment of the invention, binding entity 112 may be any molecule, macromolecule, biomolecule, material or composite which is capable of specifically recognizing analyte molecules 104. Some examples of binding entity 112 are aptamers, nucleic acids, oligonucleotides, polymers, imprinted polymers, antibodies, enzymes, proteins, Fabs (antigen-binding fragments) or phage displays.

In another embodiment of the invention, magnetic beads 108 may be any type of magnetic, ferromagnetic, paramagnetic, superparamagnetic, superferromagnetic, particles or nanoparticles or large magnets made of any magnetic material. Magnetic beads have high surface areas per unit volume, good stability, and enable fast kinetic processes involving solution species compared to bulk solid surfaces. A great advantage of magnetic beads or nanoparticles, as opposed to non-magnetic nanoparticles, is their ease of manipulation with simple, inexpensive magnets. Very efficient isolation of analytes from liquid samples can be achieved inside or outside of the detection system, so that detectors can be versatile and need never be exposed to the complex liquid sample matrix.

In a particular embodiment, ferromagnetic particles are used for detection of analytes. Unlike paramagnetic particles, such as iron oxides, the ferromagnetic particles of an embodiment (for example, neodymium magnet (NdFeB) particles) can easily undergo temporary or permanent magnetization after exposure to an external field. This enables the direct quantification of the number of the particles without the necessity of utilizing a secondary process for quantification. Moreover, since they generate no magnetic forces prior their magnetization, the use of the ferromagnetic particles solves the problems of coagulation and aggregation observed during the use of paramagnetic particles. Additionally, ferromagnetic particles may be coated with any material that protects the particles from aggregation. An additional benefit of such coating is that after the magnetization has occurred, these particles, which are natively repulsive to each other, become attracting each other and forming aggregates. Such aggregates are much easier to isolate and quantify by measuring their weight, magnetic field, light emission intensity, the amount of force they apply when pressing against a surface connected to a force measuring device under exposure to large magnetic field that propels the particles.

Using the ferromagnetic particles, which have intrinsic or extrinsic parameters, in a method of an embodiment results in only temporary magnetization. This can allow recycling of the beads and reattaching them in the immunoassay format for another round of sensing. This can also be used to develop a multiuse sensor by performing temperature, ionic concentration of pH change cycles to separate the binding entities from the analyte or analogues. During such recycling, a magnetic filter, such as an electromagnet, can be used to ensure that the magnetic particles do not leave the system, while the fluid containing an unwanted analyte, which is not supposed to be further recirculated in the system and detected, is discarded. After a certain interval of time, which is sufficient to remove the unwanted analyte, the binding conditions can be reset to allow binding of the functionalized analyte-analogue to the binding entity. After another certain interval of time, which is sufficient to allow rebinding, the system can be reused as a sensor again.

Magnetic beads 108 used in the present invention may be commercially available or prepared in the lab. According to a particular embodiment of the present invention, magnetic beads 108 are ferromagnetic beads, which are the most useful for systems requiring magnetic separation and transport as they become magnetic in an applied magnetic field, but have zero magnetization in the absence of a magnetic field. These beads are often called “super-paramagnetic”, while ferromagnetic beads feature permanent magnetism after they are exposed to applied magnetic field.

In yet further embodiment of the invention, magnetic beads 108 are paramagnetic beads, which are the most useful for systems requiring magnetic separation and transport as they become magnetic in an applied magnetic field, but have zero magnetization in the absence of a magnetic field. These beads are often called “superparamagnetic”, while ferromagnetic beads feature permanent magnetism after they are exposed to the applied magnetic field. The most common examples of paramagnetic beads have magnetic iron oxide cores and non-magnetic polymer shells featuring surface chemical functionality for attachment of binding entity 112. The magnetic core may also consist of a collection of paramagnetic nanoparticles embedded in a polymer core. Beads with sizes in the range of 100 nm to 100 nm in diameter are commercially available with variability in size <±5%. Suppliers include Solulink, Invitrogen, Bangs Labs, Merck, and others. Bead size determines sedimentation rate and mobility in a liquid flow. The outer polymer shell serves to add surface functional groups to the bead and protects the metal oxide core magnetic core from external media. The outer shell may also consist of agarose, cellulose, porous glass or silica.

The magnetic beads are also available with the surface molecules such as streptavidin, biotin, protein A, protein G, IgG, IgE and IgM. The beads pre-coated with streptavidin may capture the biotin-labeled binding entities. Protein A coated surface may selectively bind to Fc regions of antibodies for orientated immobilization.

Superparamagnetic beads are commercially available with coatings of either organic functional groups to attach biomolecules like antibodies and enzymes, or pre-coated with biomolecules that can bind specific partners.

According to a further embodiment of the invention, magnetic beads 108 are magnetic beads with sizes in the range of 1 nm to 1 mm in diameter, having the polymer-embedded iron oxide nanoparticle cores. Such beads with paramagnetic nanoparticles embedded in a polymer core matrix are superparamagnetic, but may feature multidomain magnetic structures with remnant magnetic moment. They show some degree of magnetic clustering in liquids due to induced magnetism in neighboring particles.

In a further embodiment of the invention, magnetic beads 108 are coated with binding entity 112. Since binding entity 112 specifically recognizes molecules 109, magnetic beads 108 may be attached to the inner walls, matrix or surface of cartridge 106 in the reaction between molecules 109 and binding entity 112. This is the step of the cartridge 106 preparation.

When analyte 104 passes through cartridge 106, it binds to binding entity 112, thereby replacing molecules 109 and releasing magnetic beads 108 from the cartridge into the flow. Magnetic beads are then separated from the flow by an applied magnetic field to be positioned just above magnetometer 110. When magnetic beads 108 reach magnetometer 110, signal proportional to the amount of the beads is read out. This amount of beads 108 is in turn proportional to the amount of analyte molecules 104 in the flow and the amount of time the process was taking place and sample flow rate. Magnetometer 110 may be any commercial magnetoresistive, Hall Effect, coil-based, SQUID, or any other type of device, which is capable of sensing the magnetic field or properties.

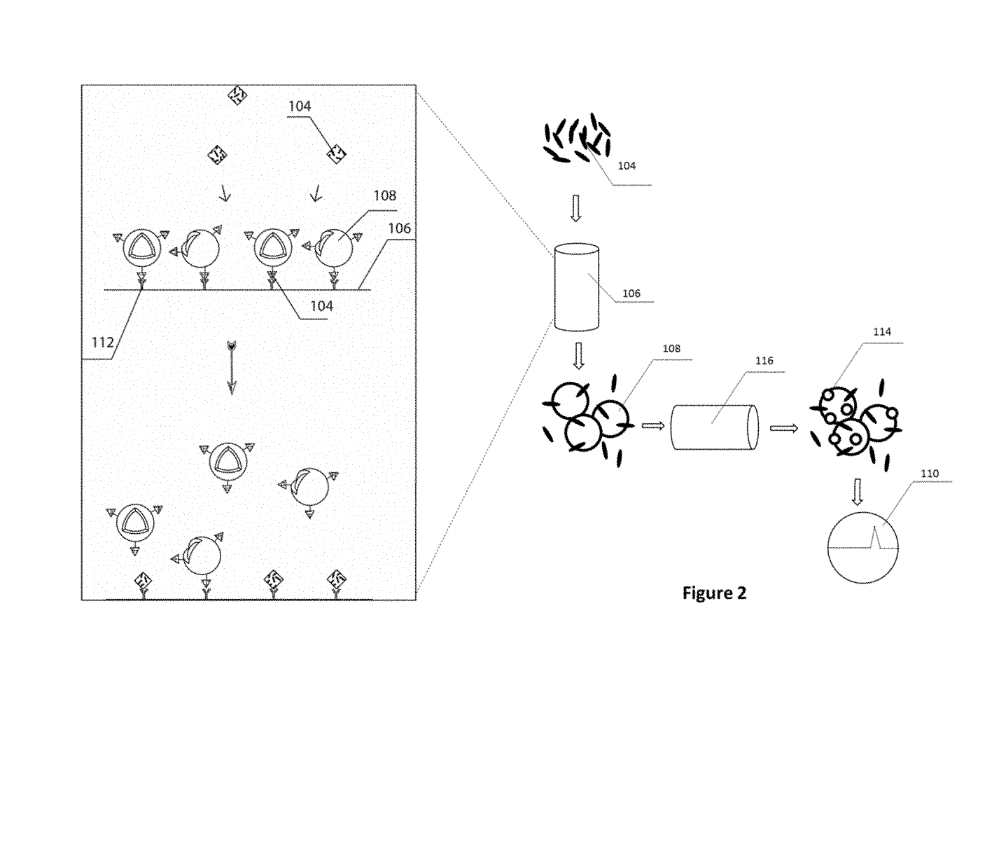

Reference is now made to FIG. 2 schematically showing the method of the present invention including a secondary amplification technique (or so called “magnetic cascade”). In one example, one or more magnetic cartridges 116 are introduced in the system for the purpose of signal amplification. In the example, magnetic beads 108 passing through magnetic cartridge 116 after being magnetized by the magnetic field generator, cause the release of additional magnetic beads 118, which amplify the signal registered by magnetometer 110. The quantity and the amount of magnetic beads 108 allowed to remain in cartridge 116 is proportional to the amount of magnetic beads 118 that are released.

In addition, left pane or inlay in FIG. 2 shows the example when magnetic beads 108 are coated with molecules 109, while binding entity 112 is immobilized on the walls of cartridge 106. In this case, analyte 104 from the flow will bind to binding entity 112, thereby releasing magnetic beads 108 in the flow. Amount of the released beads is proportional to the amount of the analyte in the flow. In this case sensitivity may be higher because no free binding sites on the magnetic beads would exist in such case, where the beads are functionalized with binding entities.

In yet another example, the additional magnetic beads 118 can reside in any suitable place in the same cartridge where the reaction takes place, such as the bottom just before the outlet, where the magnetic beads released by the analyte molecule can trigger the release of more magnetic beads through various reactions such as physical, chemical or enzymatic reactions. The beads can also reside mixed with the assay beads for easier, immediate access and interaction while the bead are still on the surface and not yet suspended in the flow which may make the chances of them interacting with the secondary beads lower. In this form, cascade will be initiated immediately upon primary beads release. In a specific case of the enzymatic reaction, the released beads are functionalized with an enzyme that can degrade a linker of parking magnetic beads causing their controlled release. Cascading beads may or may not include enzymatic element of their own that may further amplify the cascade in an exponential rather than linear manner. In another example, primary beads that are released in the competition assay are magnetized and let to flow in channels or microchannels with secondary beads residing within. The presence of magnetized beads will cause the controlled released of these beads depending on the exposure time and the amount of primary beads. In another example, both primary and secondary beads can be let to flow again into the same channels, microchannels or chambers, or into another third and fourth chambers to release more and more beads in the magnetic cascade.

One of the advantages of the magnetic cascade system is in that the additional magnetic beads are not dependent on the equilibrium created in the system and cannot be randomly displaced. Thus, the beads of any size can be used in the system to optimize the amplification of the signal. Having very large beads, even in the range of millimeters, can have a huge advantage as it allows for a very easy detection using different techniques. This is another advantage of the method of an embodiment, as until today, beads no larger than approximately 3 microns can be used in bioassays. The field of biosensing using large beads have never been suggested before, because the large beads allegedly have no apparent advantage. However, since the beads are directly quantified without using a secondary label, there is a huge advantage in a method of an embodiment of having the magnetic signal from each bead proportional to the power of three of the volume. The larger the beads, the stronger the signal, which is exponentially amplified. Moreover, ferromagnetic beads, such as neodymium magnet (NdFeB) beads or samarium cobalt beads or any other ferromagnetic alloys, are natively capable to generate much higher magnetic field than the one generated by paramagnetic particles.

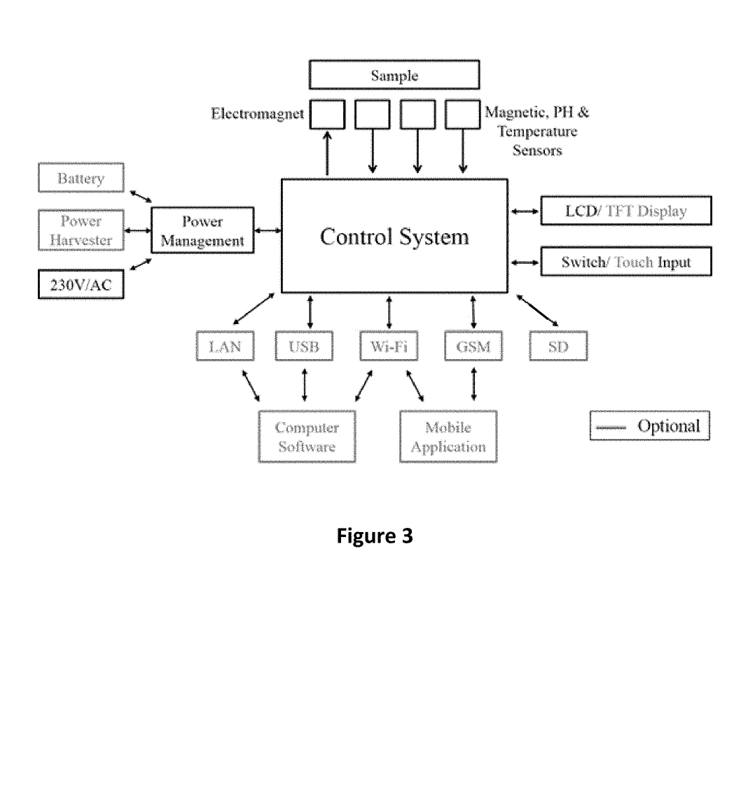

Reference is now made to FIG. 3 which shows a block diagram of an exemplary system consisting of a central core that may be a microcontroller responsible for coordinating the function of an electromagnet used for trapping and magnetizing the magnetic pollutants and released magnetic particles, and can also receive and manipulate data from other sensors measuring magnetic field (counting released beads), pH, ionic concentration and temperature of the sample (calibrating to the changing binding coefficients). It also handles data processing and automated conversion based on stored calibration table and outputs data to a LCD display or transmitted to external computer or mobile devices via USB, Wi-Fi, GSM for further processing, user alerting or storage. An external energy harvester, such as solar panel, turbine and vortex induced vibration that harvest energy from the surrounding flow may be used to produce a completely autonomous system that can function for long times in remote areas. The use of multiple cartridges that are hibernated (by freezing, for example) to increase their lifespan can allow even longer lapse of time without human intervention. Only one cartridge is used until it expires, whereupon another cartridge is being automatically commissioned. The system may also be adjusted to perform auto calibration by deliberately exposing itself to known amounts of analyte or in many similar ways at required intervals of times. To prevent false positives and false negatives, the system can include multiple cartridges that complement each other's signals. Another optional way to allow better sensitivity is to ensure that the media conditions are strictly monitored. Anti-bacterial, omniphobic coating is applied to the internal surface of the device using adhesives to bind superhydrophobic NPs for example. This technique allows saving energy, driving the fluid, especially in microchannels, and also preventing microorganisms' growth. Antibiofouling agents can periodically or constantly be added into to the system, and the system's pH, flow rate, ionic concentration, internal pressure and temperature can be controlled to allow more accurate quantification of the analyte.

In another embodiment, the magnetic beads can be used for time-depended signal sensitivity. In contrast to all prior art techniques, which process finite and usually small amounts of samples, the method of an embodiment makes it possible to continuously input a sample. The continuous testing of the sample interacting with the binding entity results in beads displacement over time that is proportional to the amount of analyte in the sample. Unlike prior art techniques, which require ultra-sensitive transduction systems in order to quantify very small amount of analyte, the method of an embodiment makes it possible to amplify the signal over time by collecting more and more beads until the signal levels are large enough for even raw transduction units to read it without error. The signal reading can be correlated with the sample flow rate and with the amount of time the process was allowed to take place, to the original amount of analyte in the sample being tested. Thus, the method of an embodiment results in an unprecedented accuracy without the necessity of using a complex transduction unit, by collecting beads and amplifying the signal over time.

### EXAMPLES

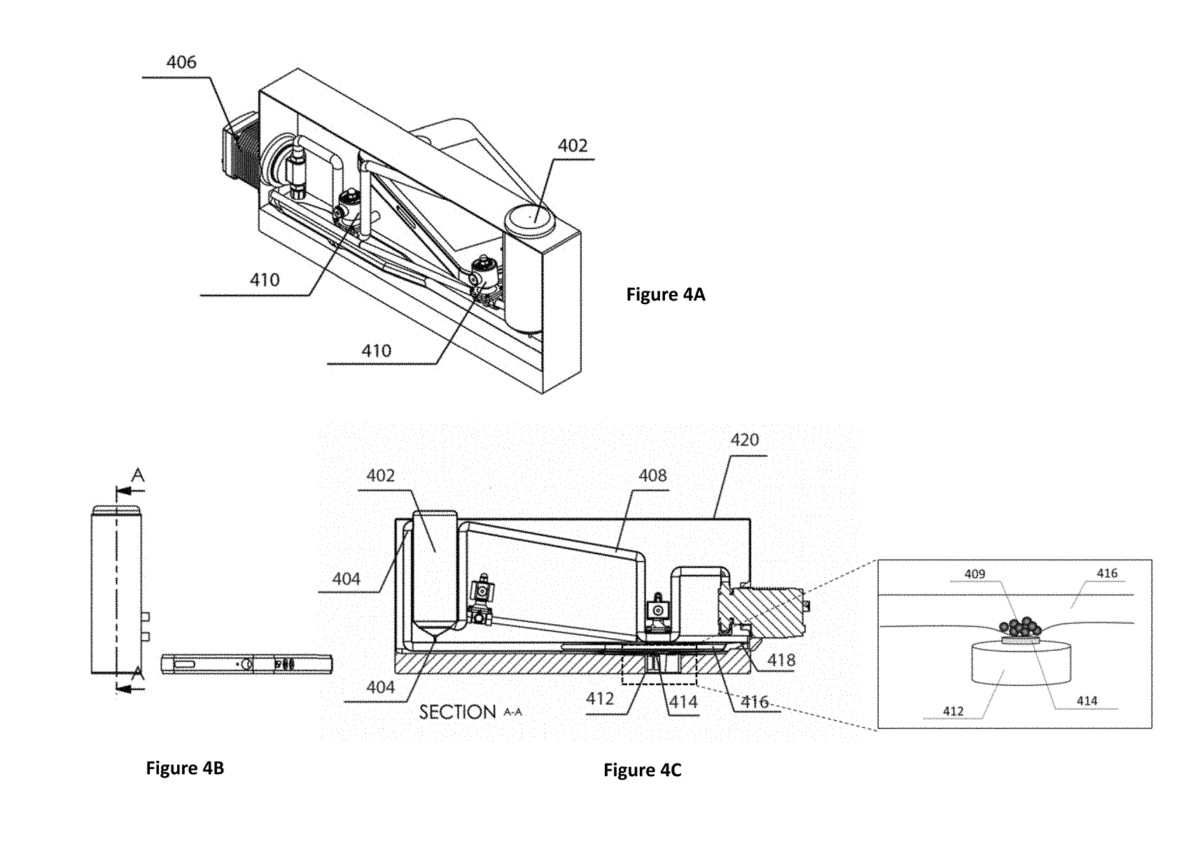

Reference is now made to FIGS. 4A-C showing the first exemplary sensing device for use in a method of an embodiment of the present application. The device is placed in housing 420. Flow enters the device through the inlet, located at pump 406. The inlet may contain filters for solid particles present in any sample effluent and may also contain a solids shredder and water injection and mixing channel before the filter for cases where the sample being diagnosed is a solid sample, for example. A condenser or a device such as gas-to-fluid flow exchange coulomb or otherwise may be introduced if the sample being diagnosed is in gaseous form.

The flow passing through tubing 408 reaches the first valve 410 mounted just after the flow passes above the magnetic field generator 412. The magnetic field generator is kept turned-on until the signal readout moment, or in another case the magnetic field generator can work in a waveform signal which oscillates from plus to minus and the signal is taken when the field strength is zero, thereby separating any residual magnetic material from the flow that was able to elude the initial filters. The separated magnetic material is collected, and can be further washed away from the device through outlet 418.

Sample continues to flow through tubing 408 and enters immunoassay cartridge 402, where the reaction takes place (as explained above). As a result of the reaction, the magnetic beads are released into the flow, leave cartridge 402 via outlets 404, pass through tubing 408 (where the tubing cross section may become wider in order to slow down the beads) and collected in the chamber 416, which is designed to control the flow, in order to maximize the collection by magnetic field generator, reaching magnetic field generator 412 where they are separated from the flow as they're being trapped and magnetized by the magnetic field. After that, magnetic field generator 412 is turned off, and the signal from the magnetic beads is registered with magnetometer 414. The signal is proportional to the amount of the magnetic beads released, and in turn is proportional to the concentration of the analyte in the sample. The longer the time the beads are collected, the higher the signal and the latter can be interpolated back to indicate the original concentration of analyte in the flow. This time-dependent signal amplification is controlled by the user which is able to take the measurement at any time depending on the level of sensitivity the user requires. One of the embodiment of the present invention is that there is no sensor today that would allow an infinite signal amplification such as the one described above. Normal amplification today is done using a secondary amplification reaction, however the use of the micro magnets in the present invention enables this unique way of single-step signal amplification, which allows the use of very cheap and simple components for detection such as magnetometer or even scales.

When using the magnetometer 414, multiple ways can be used to reduce residual and ambient magnetic noise. One example is to use waveform magnetic field generated by the magnetic field generator (from +xT to −xT), is this way the signal can be taken when the magnetic field is zero, to reduce noise due to residual magnetization. Nevertheless, there are endless ways to reduce noise in this invention and the latter does not mean to confine the invention but an example only.

In yet another example, multiple magnetometers can be installed in the surroundings and measure the signal together with the magnetometer 414 to subtract the residual noise from the actual signal produced by the magnetized beads.

In yet another example, magnetic shielding can be used in conjunction to any other method, to attenuate ambient fields. In an example derived from this case, a permanent magnet can also be used as the magnetic field generator to reduce power consumption. This magnetic field can be attenuated prior to signal measurement by moving the magnet away or alternatively moving a magnetic shield, such as layers of pyrolytic carbon and Mu-metal or any other, in between the magnet and the magnetometer.

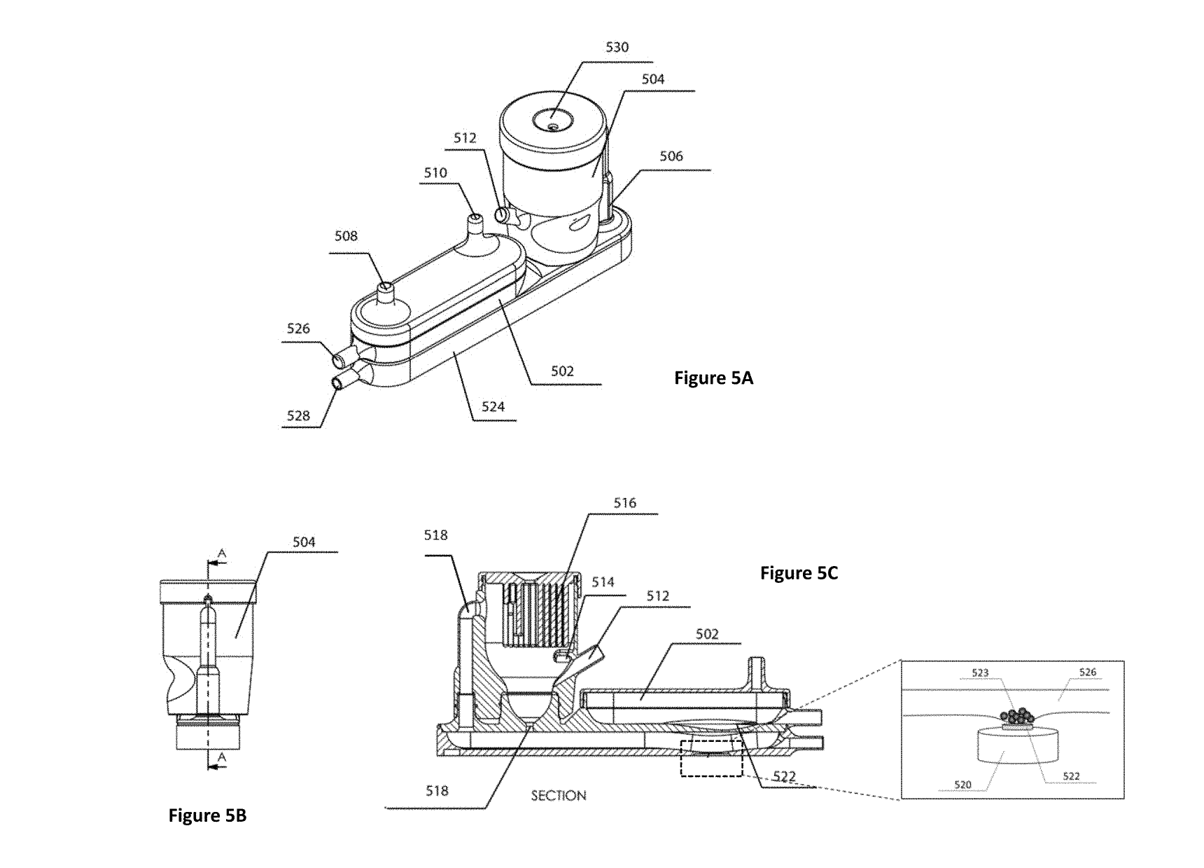

Reference is now made to FIGS. 5A-C showing the second exemplary sensing device according to another embodiment of the present invention. When the system is in use, sample is allowed to flow into upper chamber 502 through inlet 510 which is designed to slow down the flow. Magnetic field generator 520 that is kept-on during normal operation is used to separate magnetic pollutants that potentially exist in the incoming sample and may disturb accurate measurements later-on.

After predetermined amount of time, the magnetic field generator is turned-off and valve is opened allowing to wash the magnetic components that were previously trapped away from the device through outlet 526. Otherwise, water flow that may be containing the analyte is pumped through outlet 508 toward inlet 512 of cartridge 504 through tubing (which is not shown in the figure and where ambient parameter measurements may take place). Cartridge 504 is optionally equipped with carefully designed preservative magazine holder 530, which can optionally release anti-biofouling substance, to increase the lifespan of cartridge 504 (preservative can also be released in any other location in the system such as the main inlet or chamber). The reaction described above takes place on reaction substrate 516 which is designed for maximum diffusion rate and low shear forces and is containing the analyte-analogue molecules complex inside cartridge 504. As a result of the possible competition reaction with present analyte, the magnetic beads with the newly bound analyte (or bond analogue in cases when the binding entities are bonded to the matrix) are released, then passed through outlet 518 into lower chamber 524 which is designed to slow down the fluid velocity, and finally collected by magnetic field generator 520 which may be located below the magnetometer and is designed to concentrate the beads directly above the magnetometer for more accurate reading of the sample. Magnetometer 522 can read the signal at any time specified and the signal read will be proportional to the amount of magnetic beads collected which is proportional to the concentration of analyte and the period of time the bead collection was made. As above, the recorded signal is proportional to the amount of the magnetic beads collected from the flow, and in turn is proportional to the concentration of the analyte in the water effluent. Optionally, the device can be equipped with flow control device 514 that further improve the flow patterns and prevent shear forces on the surface of 516. Device outlet 528 is used to discard the sample effluent from the device.

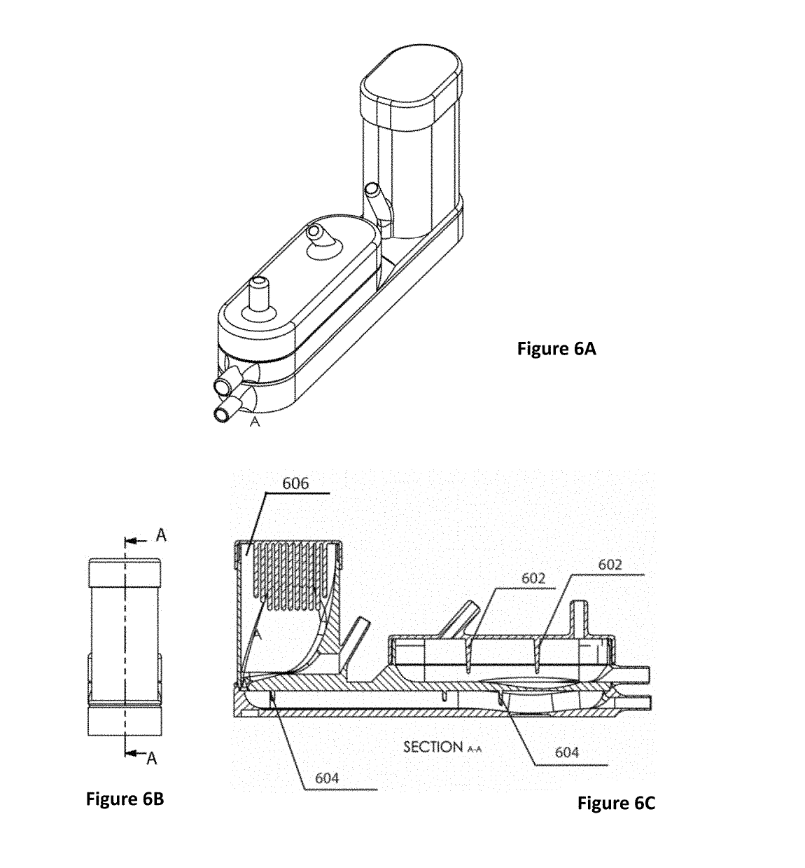

Reference is now made to FIGS. 6A-C showing the third exemplary sensing device according to yet another embodiment of the present invention. The main difference of this device compared to the device shown in FIGS. 4A-C is in the instillation of one or more flow control devices 602 and 604 installed in the upper and in the lower chambers, respectively. Control device 602 installed in the upper chamber is used to redirect the flow patterns and increase particles retention time (by formation turbulences and forming pseudo chambers) and forcing magnetic pollutants, potentially present in the sample, to stay longer in this filtering chamber and closer to its bottom where the magnetic field is stronger and cavities to trap the particles can also be installed, thus maximizing the collection and filtering effects of the magnetic generator, to ensure the maximum number of the magnetic components polluting the sample are separated before the sample entering reaction cartridge. The top of this filtering chamber, or an additional separate chamber can be equipped with a filter to trap and eject suspended solids that can also be an interfering factor.

In order not to have the filtering unit at all, saving costs when manufacturing smaller devices for example, the magnetic filtration unit can be completely removed and instead blank sample that does not interact with the cartridge can be analyzed by the system to have a reading of the amount of magnetic pollutants natively present in the sample, a value that can be removed later on when measuring for the amount of analyte.

One or more control device 604 installed in the lower chamber is used to redirect the displaced magnetic beads towards the magnetic field generator, to ensure the better collection targeted to be above the magnetometer before the signal readout. Another thing that is new about this specific implementation is that there is only one outlet in the reaction chamber 606. This results in virtually no flow shear forces near the substrates where the magnetic beads are preventing undesirable magnetic beads removal due to flow forces. It is important to mention however, that shear forces may be wanted in some implementations where the beads are held by multiple bonds. In these cases, shear forces are needed to displace the beads once a number of these bonds was incapacitated due to displacement or other reaction of analyte present in the sample. Other reaction can by enzymatic cleavage for example, of a crosslinker due to activation by a present analyte. Thus it is important to emphasis that displacement, competitive, sandwich or any other immunoassay form that may be implemented in this system is only an example, the release of beads due to presence of analyte can be done in various way that were previously mentioned prior arts and many ways that will become obvious or be developed specifically in order to the release mechanisms for the new systems described here.

Another implementation of the system and method that is presented here is an “upside down” approach which is having beads flowing around and being observed for example, by binding in sandwiched format or due to surface or chemical modification done due to presence of analyte. This causes the removal of beads from the flow and lower magnetic signal generated upon direct measurements of magnetic field or by a similar process to processes described here. This will also include a filter, either size-based or magnetic- or affinity-based to prevent escape of beads from the circulating flow which is continuously replaced in order to continuously measure a new sample.

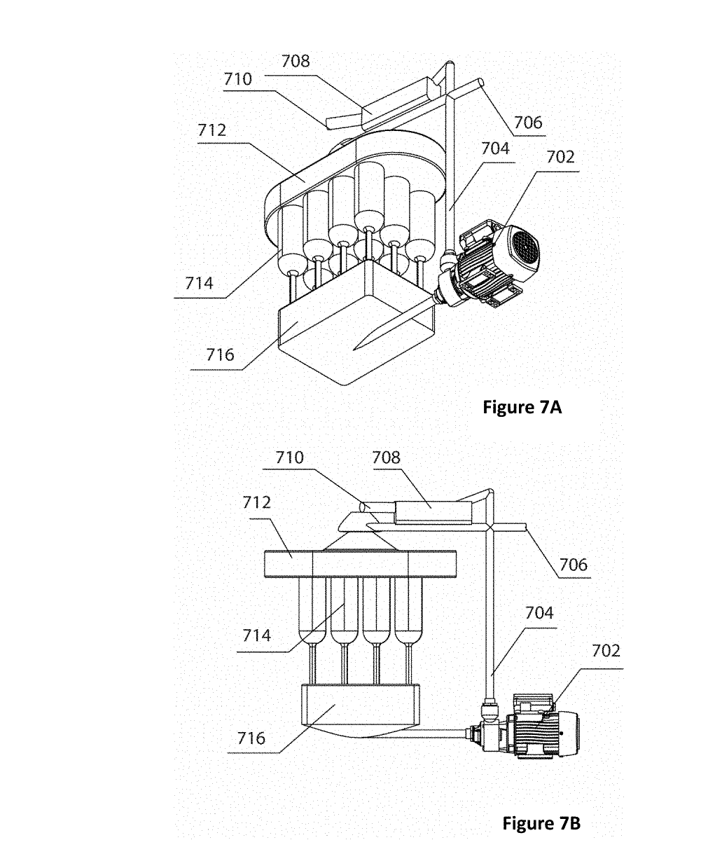

Reference is now made to FIGS. 7A-b showing one optional configuration of a sensing apparatus for simultaneous sensing of multiple analytes, which all the example configurations can be in this form. The sample will flow in from the pump 702 through tubings, in one example, sample will enter from 706 and pass several closed loop to prevent losing magnetic particles. In another example. One or more pump may be used to transfer sample solution from inlet 710 and 706 into chamber 712 which distributes the sample into separate sensing channels. Samples that have flown through 704 are collected in 716 and may be pumped to flow through 708 and magnetic beads can be collected in a secondary chamber 708 for reuse.

The above example is only a narrow, specific possible implementation of the said multi-analyte system. This can be done by any means and for example having all or groups of analytes (detection reagents) in separate detection cartridges to be circulated separately and then transferred one by one to the magnetometer for measurements. Alternatively, the system will include one input inlet that is funneled at each time period to a separate cartridge resulting in a system that takes measurement for each analyte one after the other in repeating cycles. Alternatively, for systems when speed is more crucial than cost, each cartridge may contain separate separation and/or detection unit for detection of all the analyte or groups of them—simultaneously.

A device, system and method in accordance with some embodiments of the invention may be used in many variations and different forms, for example, in conjunction with a device which may be built in a lab-on-chip style using microfluidics and allowing multiple analyte sensing configuration.

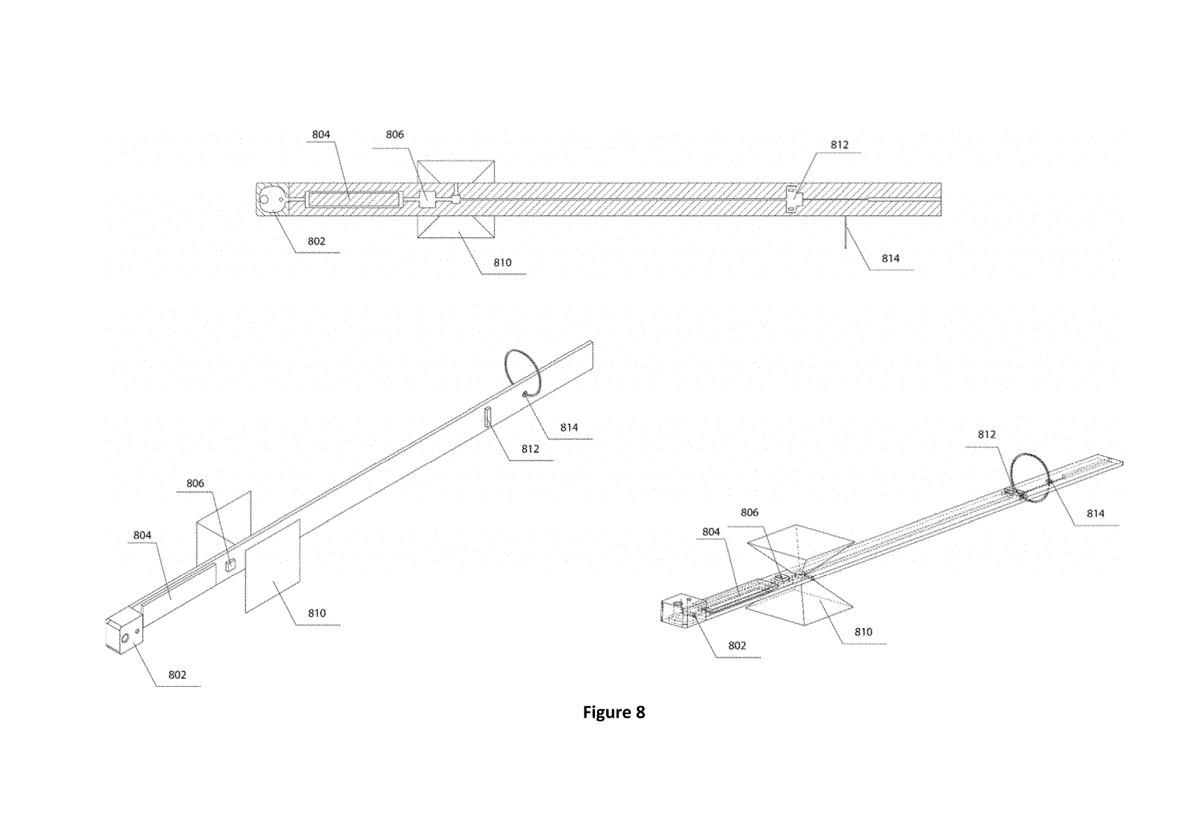

One example is illustrated in FIG. 8, wherein the sensor is realized in a microfluidics scheme. For explanation purposes, the analyte to be tested is mRNA as an indicator of a presence of a certain bacterial strain and determination of its viability in a solid food sample. The sample first entered into the system via a sample homogenizer 802 which crushes the sample and dissolve it in water. The homogenized sample is then sucked into chamber 804 in which it resides for some time. In this chamber, physical properties of the sample are measured and adjusted by various sensors, such as temperature, turbidity, pH, ionic strength, flow rate, pressure and other additional sensors and controllers. For the purpose of amplifying and distinguishing mRNA signals of viable over non-viable bacteria, the chamber may also include transcription factors that induce over-expression of the specific mRNA analyte. Moreover, chemicals which selectively lyse only non-viable bacteria can also be introduced. In addition, ssRNAses can be tethered onto inserted beads or inner surfaces of the chamber to eliminate any analyte mRNA that may be present due to non-viable bacteria. After a few minutes of the content of chamber 804 is passed into chamber 806 where viable bacteria are lysed and all other organizes are killed. This can be achieved for example by rapidly circulation abrasive beads which shreds the membranes of any living cell. Optionally, a quick polymerase chain reaction can be performed to further amplify the number of analyte mRNA. Next the sample is flowed into a chamber which filters any magnetic impurities that may have been present in the sample. This can be achieved, for example, by placing a pair of pyramid N52 magnets 810 connected to each other by iron rod. If the gap between the tips of the pyramids is about a millimeter, the magnetic field to which the flow is exposed can easily surpass 2T, effectively capturing all but the most elusive magnetic impurities. Subsequently, the flow reaches the immunoassay cartridge chamber 812 in which the assay takes place and beads are displaced at a rate that is proportional to the amount of mRNA analyte. Specifically, to RNA or DNA, the displacement can be achieved either by one-to-one replacement of the complementary strands, or preferably by attachment of the incoming mRNA to a complementary DNA or RNA strand that is used to hold a bead. By this attachment, part of the strand that is holding the bead is converted from double strand to single strand. By introduction of enzymes which selectively cleave only double strand RNA, DNA or DNA-RNA—the link between the bead and the surface will be cleaved, effectively releasing the bead into the flow. Subsequently the rate of beads release can be measured by a sensor 814, which measures properties of the incoming beads, such as their magnetic properties.

In addition, the disclosed assay may be combined with any labeling technique, for example fluorescence-based technique. The device itself may contain additional components, for example temperature control unit or refrigerating unit using thermoelectric plates, compressor for the main cartridge, part of the cartridge or reserve cartridge to increase the span between the cartridge replacement and maintenance. The method of the present invention may be applied, for example, for on-site testing of water in water reservoirs or water plant effluents, pharmaceuticals production, food production, even gaseous or solids production by transforming the samples into liquid sample by a verity of possible means. It can also be used for on-site testing of explosives in airports, or on-site testing of food products, point-of-care blood and other medical diagnostics or even personal monitoring for food or liquid safety. This is of a particular interest as a personalized device for detection of allergens, pathogens and toxin does not exist today as it will be impractical to even conceive it due to cost and training needed by the user. However, the device described here does not require any user training and may even by left by itself inside a food package or at the refrigerator at home. User can easily take a piece of their sample (food for example) and place it in the device which, within minutes or even seconds can report if it is adequate for use. As the machine is cheap and the detection cartridges can be used for a prolonged periods of time, even for multiple positive detections (as there are still large enough amount of beads will still be present), users can use it without significant cost. Cartridges containing a mixture for detection of many analytes simultaneously can be placed in one unit as the user only care for contaminated/not contaminated answer in many cases. It can also be used as an alternative to lateral strips where quantitation is needed, for example in dengue virus detection or monitoring of pregnancy or veterinary, hormonal levels, blood sugar etc. However, the scope of the present invention is not limited in this regard. For example, some embodiments of the invention may be used in “non-complete” form, for example, strips containing reagents with magnetic beads can be placed inside food package at home or at the supermarket or factory distribution or packaging stages. The beads from the displaced strip can travel to another strip to be measured by a device that is external to the food package. Similarly, the device can measure the “absence” of beads of the strip directly. Beads released can also be transferred into another strip inducing color change, electrical signal and the like rather than magnetic reading. The measurement device may be placed permanently near the package or by manually or automatically shifter from package to package. Additional conjunction with flow strip devices modified with magnetic beads of the present invention is an option. Such flow strips may be inserted, for example, into food packages. If the specific analyte is present in the food product, the displaced magnetic beads will be removed from the strip and detected with a magnetometer placed near the food package to magnetize the displaced beads.

In another implementation this new magnetic detection technique may by combined with other techniques such as fluorescent to give multidimensional information regarding a sample. For example, magnetic beads measured an also contain a specific fluorescent label that indicate the presence of another analyte. In this form, for example, beads for detection of a specific bacteria can be also functionalized with and linked with more specific strain antibodies that will be color-dependent. Beads can contain two or more sets of binding entities for that are needed for their release giving quantification of two separate elements in a single detection. This can be further expended by utilizing fluorescent markers of various colors to provide information on a matrix of parameters with a single detection step. It can also be further expended as cellular elements for example can be pre-labeled with fluorescent markers that are protein- or trait-specific to provide abundant of information on the detected sample. Fluorescent is not the only secondary label possible, the beads, their linkers and the surface may be functionalized with a verity of different elements that may provide additional sample and more specific and accurate analyte information. Specific membranes and filters may be placed to collect beads with markers and labels that are label-specific. In general, this technique may be combined with a verity of other techniques that are available today or may be specifically developed in the future.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### Synaptic Sensing

Another means by which one can construct the suggested sensing scheme resembles the function of a neural synapse. The balance of forces acting of the beads to be displaced are categorized into two groups: balancing forces and unbalancing forces. Among the balancing forces can be Brownian motion, gravity, flow forces, inertia or centrifugal forces the may arise from vibration or rotation of the matrix onto which the beads are attached, magnetic forces or other forces that may be applied to detach the beads from the surface. In contrast, the forces that acts to bind the beads to the matrix can be antibody—analyte bonds, or other bonds between the recognition element and the analyte, ionic forces, electrostatics interactions, hydrogen bonds, van der Waals interactions, capillary forces and other binding forces that can act to bind the beads to the matrix.

As long as the binding, or balancing, forces are stronger than the forces that push towards detachment of the beads are stronger the bead will remain attach to the matrix. However, once the unbalancing forces surpass the balancing forces the beads will be displaced. In order to maximize sensitivity of the system, it is prudent to design the system in such way that the number of recognition elements—analyte bonds are minimal. Thus, by disconnecting a minimal amount of these bonds, the unbalancing forces will surpass the number of balancing forces and the bead will be displaced. Ideally, to receive the maximum sensitivity from the system, the threshold for such displacement should be a release of one recognition element—analyte bond. However, if a release of a single bond results in the immediate displacement of the bead, there will be no safety region from false positive reports as even a random release of any of the bonds that may happen at any moment by chance, and it will cause the displacement of the beads. Thus one must engineer a safety range wherein spontaneous release of bonds does not lead to the release of the bead.

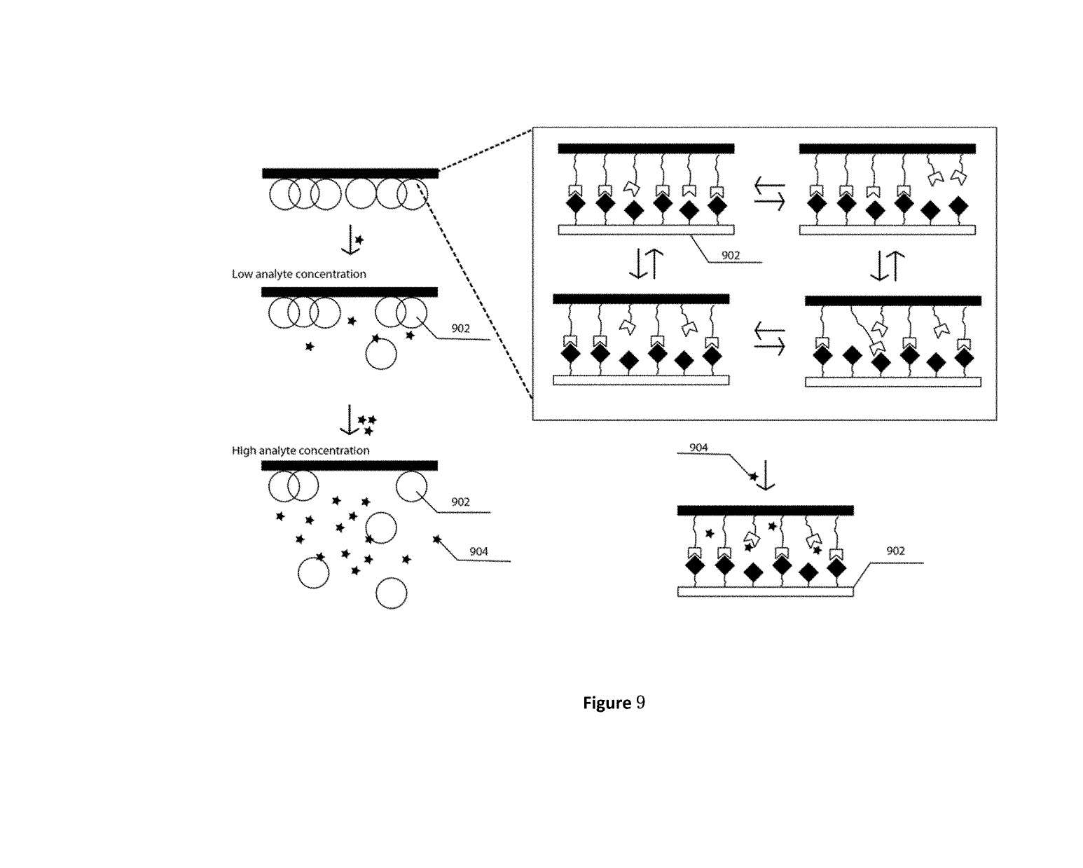

Furthermore, in order to have a functional beads displacement system that can work for all types of analytes, the system must be engineered in such a way that it fluid. Meaning that analyte—recognition elements bonds constantly bind an unbind. This is because if bonds are fixed without spontaneously releasing from time to time, like it shows in the top right four images in FIG. 9, it will be impossible for a third party analyte 904 that may be present in the solution to occupy the binding site on the recognition element. Thus, one can say that a system in which the rate of unbinding and rebinding of bonds is higher will react faster in the presence of analyte in the surrounding. In other words, it will be able to report faster the presence of an analyte. In such a system bonds are constantly associating and dissociating and the presence of an analyte in the surrounding disturbs this equilibrium by occupying binding sites, causing the release of the bead. It is thus possible to engineer the system in such a way that the binding coefficient of the real analyte is much stronger than that of the analyte mimic. In this case once the real analyte binds to the recognition element it will not dissociate spontaneously, upsetting the binding balance of the synapse between the bead 902 and the matrix even more, leading to faster displacement kinetics and response at lower concentration of analyte, as it shows in the bottom two images in FIG. 9. However, it is not always easy to engineer an analyte mimic that binds selectively only to the recognition element and still have a high rate of spontaneous dissociation. To artificially increase the response time of the system we propose induce this spontaneous dissociation by changing the conditions of the surrounding. This can be an increase of temperature, change in pH, change in ionic concentration, change in solvent, etc. We term this change a “shock”. By inducing a shock, we induce the faster dissociation of a larger number of bonds, thus increasing the kinetics by which the analyte that may be present in the surrounding occupy the bonds, hastening the displacement of the bead in the presence on analyte. One way to implement this shock is by bringing the reaction conditions just to the threshold after which the beads will displace without the presence of analyte. In this case, introducing a minimal amount of analyte will immediately cause the displacement of beads. Another type of shock can be accomplished by bringing the reaction condition below the threshold of displacement but for a very short span of time. In other words, changing the surrounding conditions to a setting in which the rate of dissociation of bonds is higher than the association rate. In this case, the beads will displace even without the presence of analyte if the change in reaction condition is permanent. The trick here is to engineer the shock in such a way that just before beads start to displace the reaction condition is brought back to a point where association rate is higher than dissociation rate. Thus, if analyte is present in the surrounding it will occupy some of the bonds, ultimately leading to displacement after a finite number of shock cycles.

Minimizing Binding Forces that do not Rise from Recognition Element—Analyte Bonds

As described above, if the sensing mechanism utilizes a synapse-like scheme in which multiple recognition elements—analyte bonds are being used to hold a single bead, the system is greatly depended on the balance of forces attaching a single bead to the matrix. In order to minimize electrostatic interactions between the bead and the matrix, one can install a fluffy layer of perfluorocarbon or fluorocarbons on the surface. These fluorocarbons can be elongated chains that are linked in one side to the matrix and are having a free end. Such chins limit electrostatic interaction with the matrix. A certain number of these chains may have functional elements rather than free ends which will allow the connection of recognition elements or analytes to them to enable the displacement scheme. The beads on the other end may also be covered with said perfluorochains. However, in order to increase the affinity of the beads to the aqueous solution and decease its affinity to the surface of the matrix, the use of polyethylene glycol chains or other hydrophilic chains can be utilized. Other combinations of surface modifications can be utilized to achieve similar effects.

### Specific Application Using DNA

For detection of DNA, or in cases where the analyte is bacteria and there is importance in the identification of live cells or bacteria, PNA/DNA/RNA can be used to form a displacement scheme.

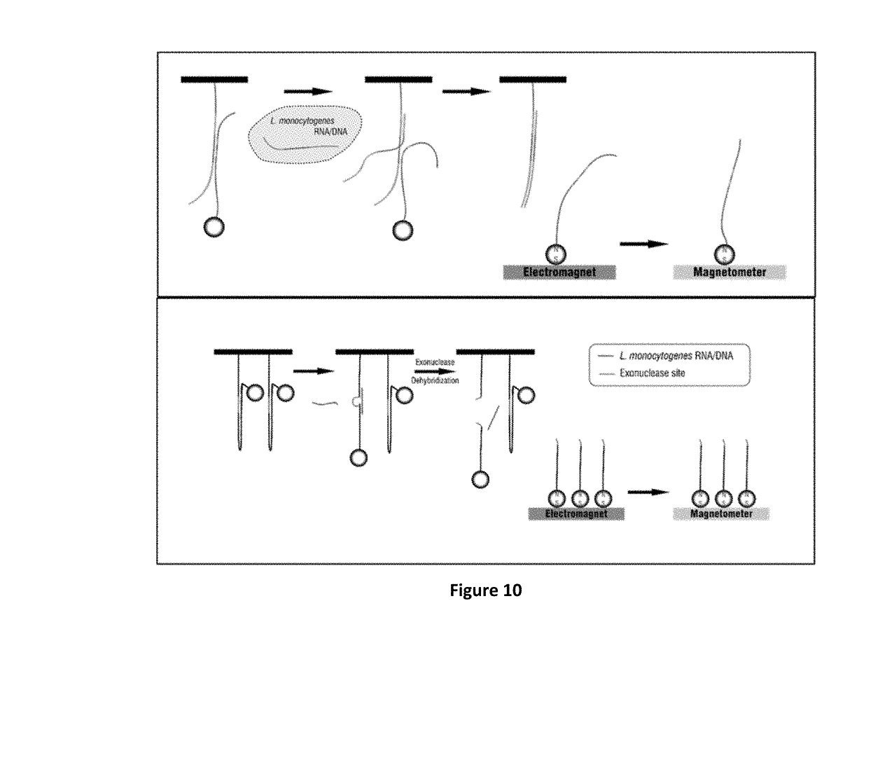

DNA/RNA sequences can be identified FIG. 10 (top). Here, capture DNA on a substrate hybridizes to a specially designed partially complementary strand (with deliberate base modifications, mismatches, additions or deletions) on micromagnets. Presence of sequences of analyte RNA/DNA that are fully complementary to the capture DNA strands will result in the displacement of the partially complementary strand, thus releasing the beads.

In another scheme, a DNA/RNA reporter can be used (see FIG. 10 bottom). The reporter which may be a PNA (Peptide Nucleic Acid) binds to DNA/RNA analyte to reveal a restriction site recognizable by an exonuclease which in turn cleaves the reporter, thus releasing the bead. By repetitive cycles of hybridization and dissociation analyte DNA/RNA can repeatedly cause the displacement of beads that are collected and measured. The technology differentiates itself from real time-PCR in that it does not require actual DNA replication and uses much simpler magnetic-based transduction. Thus, it does not require bulky, expensive optical components. Furthermore, the use of PNA allows higher specificity, improved binding kinetics and robustness and strength and is resistive to enzymatic degradation from biofilms. Thus, unlike PCR diagnostic techniques, PNA will allow for a robust system that can operate in continuous mode and in the presence of interferants. This approach allows analyte DNA/RNA to release multiple beads thus achieving additional amplification effect.

By using RNA detection, it is possible to distinguish between live and dead cells as mRNA have a short livespan, this can be further amplified by first exposing the cells to a solution with conditions that hasten the degredation of mRNA. Subsequently, after eliminating mRNA that might have been present in dead cells, elements that amplify the expression of the target mRNA being detected can be introduced to the solution to force only living cells to over express said mRNA analyte.

Following lysing of the cells will result in release of the mRNA analyte which in turn can be used for the displacement of the beads. the scheme described above in which live cells can be distinguished from dead cells is not limited to mRNA detection. Artificially, one can utilize cell transductions pathway to cause the over expression of a certain protein which will be a selective indicator for a live cell. Moreover, one can utilize cell lysing techniques prior to detection stage which are selective to dead cell, being harmless to live cells which can further improve the detection selectivity of the system.

### Direct Cleavage

In another embodiment of the invention, a system does not comprise of recognition elements—analyte bonds to hold the beads to the matrix surface. Rather a linker is used to directly link the beds to the surface. This linker is susceptible to cleavage by either the analyte or a third party that is activated by the analyte. If mercury is the analyte for example, the linker can be a molecular chain that is cleavable by an enzyme that is activated selectively by mercury.

### Double Strand Cleavage

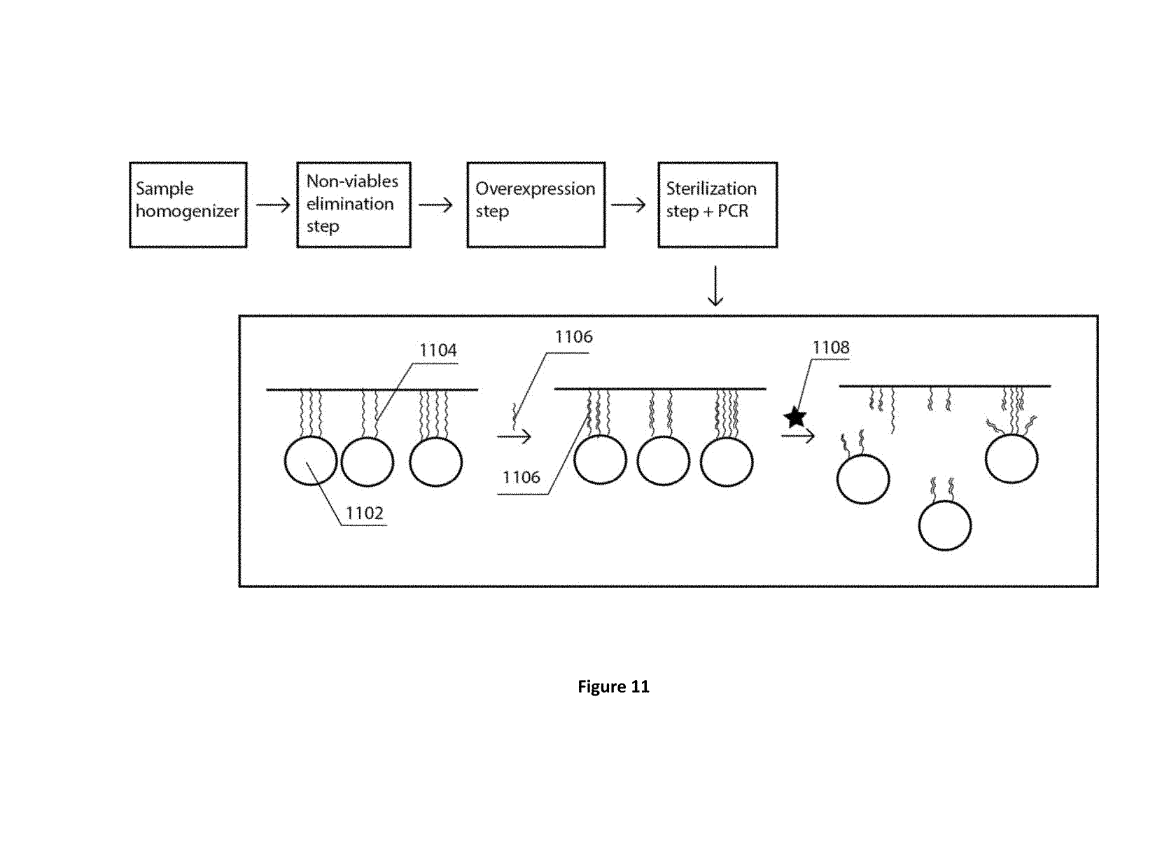

In another embodiment of the invention, beads are held to a matrix by single strand ribonucleotide chains 1104 (such as DNA, RNA, LNA, etc; for the sake of simplicity we will describe the system as an RNA-based system, however if can be achieved with DNA, etc) as shown in FIG. 11. A portion of each of these chains that are holding the beads 1102 contains a complementary sequence to an RNA or DNA sequence that is selective to the analyte. Thus, when the analyte is present, its RNA 1106 that is released to the surrounding by lysing the cell or by forcing it to secrete RNA can be made to hybridize to the single strand chains that are holding the beads. Enzymes, temperature, etc. can be used to ensure that the RNA is hybridized very selectively and that partial hybridization are minimized. signals and environmental conditions can be used to force the analyte to overexpress the RNA chain that is being targeted.

Consequently, the formed double strand RNA chains are exposed to a dsRNAse 1108, an enzyme that selectively cleave and degrade double strand RNA chains. Thus, only when analyte RNA is present, a segment of the chains holding the beads will be converted to a double strand chains, leading to cleavage of the chains that are holding the beads that is thus displaced and can be measured. The advantage of using mRNA is that it can easily be made to distinguish between live and dead bacteria, as live bacteria can be made to overexpress a certain sequence of mRNA that is otherwise not naturally made to high concentration. Another advantage of this embodiment is that the system can be easily and quickly adopted to detect a very large variety of different analyte at a low complexity. Polymerase system can also be introduced in order to further amplify the RNA or DNA analyte that is targeted for detection.

1. A method for sensing an analyte in a flow system using magnetic beads as sensing species, comprising the following steps:
a) pumping the flow containing said analyte into said flow system;
b) optionally filtering off magnetic impurities from the flow;
c) directing the flow into one or more reaction cartridges of said flow system for reacting the analyte with a binding entity attached to said magnetic beads inside the reaction cartridges, thereby releasing said magnetic beads from the reaction cartridges into the flow;
d) accumulating said flowing magnetic beads in a collecting area; and
e) recording a signal corresponding to the rate of the magnetic beads' release from said reaction cartridges (the amount of said accumulated magnetic beads per unit of time);
wherein:
(i) said magnetic beads are initially attached to the surfaces of said reaction cartridges via a complex with said binding entity prior to reaction of said analyte with said binding entity;
(ii) said analyte is capable of reacting with said binding entity onto said magnetic beads, thereby replacing said magnetic beads from said complex and releasing them from said reaction cartridges into the flow,
(iii) said cartridges can operate in sequence or in parallel, and
(iv) said rate of the magnetic beads' release from said cartridges is proportional to the amount of said analyte bound in said reaction cartridge.

2. The method according to claim 1, wherein steps a) to d) are continuously repeated for the predetermined amount of time, thereby amplifying the recorded signal.

3. The method according to claim 1 further comprising one or more optional secondary amplification steps or magnetic cascade.

4. The method according to claim 1, wherein said flowing magnetic beads are collected by utilizing magnetic field, physical barrier or chemical linkage.

5. The method according to claim 1, wherein said signal is recorded with a magnetometer or with mass scales mounted onto said reaction cartridge or placed in a secondary location and measuring an applied force on a magnet attracting said magnetic beads.

6. The method according to claim 1, wherein the amount of said accumulated magnetic beads is measured by using a magnetic properties sensor, said mass scales, or by applying a magnetic field to attract said magnetic beads and measure said force on a surface they apply for counting them.

7. The method according to claim 1, wherein (1) the analyte is selected from toxins, viruses, pathogens, explosives or any other ecologically, agriculturally, forensically, toxically, therapeutically or pharmaceutically important molecules; and (2) the flow is any suitable liquid, gas or air.

8. The method according to claim 7, where the liquid is water.

9. The method according to claim 1, wherein the magnetic beads are paramagnetic beads, superparamagnetic beads, superferromagnetic beads, ferromagnetic beads or miniaturized magnets, all of which can be either non-magnetized or magnetized.

10. The method according to claim 9, where said magnetic beads are ferromagnetic beads.

11. The method according to claim 10, wherein said magnetic beads comprising a magnetic metal alloy core and a non-magnetic polymer shell, wherein said non-magnetic polymer shell is suitable (1) for adding surface functional groups to said magnetic beads for protecting said magnetic beads from an external media, and (2) for surface chemical attachment of the binding entity.

12. The method according to claim 11, wherein the non-magnetic polymer shell is made of agarose, cellulose, porous glass or silica.

13. The method according to claim 9, wherein said magnetic beads are non-magnetized beads, capable of being converted to permanent micro-magnets after being accumulated in the collecting area.

14. The method according to claim 1, wherein said magnetic beads have a diameter in the range of 10 nm to 1 mm.

15. The method according to claim 1, wherein (1) said magnetic beads are coated with said binding entity and attached to the surfaces of said reaction cartridges via affinity interactions with the immobilized analyte or analyte-analogue molecules, or (2) said magnetic beads are coated with the analyte or analyte-analogue molecules, and the walls of said reaction cartridge are coated with said binding entity.

16. The method according to claim 1, wherein (1) the surfaces of said reaction cartridges are pre-activated with functional groups or functionalized with said analyte or analyte-analogue molecules, capable of reacting with said binding entity, using cross-linkers; or (2) the surfaces of said reaction cartridges are polymeric and pre-activated using carbene or nitrene chemistry.

17. The method according to claim 16, wherein (1) said functional groups are selected from carboxyl, amine, N-hydroxysuccinimide (NHS), sulfhydryl, epoxide, hydroxyl and tosyl; (2) said functional groups are activated for coupling with the binding entity using the EDC-coupling chemistry for carboxylates, or glutaraldehyde for amines; or (3) said functional groups are activated for coupling with the binding entity using surface tosyl, cyanogen bromide, NHS or epoxide groups.

18. The method according to claim 1, wherein said binding entity is a macromolecule, biomolecule or any other entity capable of specifically recognizing the analyte in the flow, selected from aptamers, nucleic acids, DNA, RNA, dsRNA cleavage system, oligonucleotides, polymers, imprinted polymers, antibodies, antibody fragments, antigens, enzymes, proteins or phage displays, or combination thereof.

19. The flow system according to claim 1, whereas said magnetic beads or said analyte are (1) modified with one or more fluorescent probes for providing further information on said analyte; or (2) functionalized with an enzyme that can degrade a linker of secondary magnetic beads.
